(19) United States
(12) Patent Application Publication
(10) Pub. No.: US 2013/0325356 A1
(43) Pub. Date: Dec. 5, 2013

(54) METHODS FOR DETERMINING HYDRATION OR DEHYDRATION LEVEL IN A SUBJECT

(71) Applicant: Cantimer, Inc., Menlo Park, CA (US)

(72) Inventors: Michael R. Elashoff, Redwood City, CA (US); James Patrick Reed, Redwood City, CA (US)

(21) Appl. No.: 13/906,168

(22) Filed: May 30, 2013

Related U.S. Application Data
(60) Provisional application No. 61/653,276, filed on May 30, 2012.
(60) Provisional application No. 61/714,716, filed on Oct. 16, 2012.

(51) Int. Cl. G01N 33/487

(52) U.S. Cl. CPC G01N 33/487
USPC 702/19

(57) ABSTRACT

Methods for ascertaining a dehydration or hydration level in a subject are described. The methods comprise determining the concentration of a salivary indicator and, in some embodiments, assigning a hydration or dehydration level based on the salivary indicator concentration after mathematical transformation of the value and with regard to previously-determined, transformed baseline values or with regard to a correlation.

### CROSS-REFERENCE TO RELATED APPLICATIONS

This application claims the benefit of U.S. Provisional Application No. 61/653,276, filed May 30, 2012 and of U.S. Provisional Application No. 61/714,716 filed on Oct. 16, 2012, both of which are incorporated herein by reference in their entirety.

### STATEMENT REGARDING GOVERNMENT INTEREST

This work was supported by Technical Support Working Group, Grant number W91CRB-080C0-0066. Accordingly, the United States government has certain rights in this invention.

### TECHNICAL FIELD

The subject matter described herein relates to methods, systems, devices, and/or computer program products to monitor hydration or dehydration levels in a subject.

### BACKGROUND

Appropriate hydration level in the human body is vital for health and proper functioning of the body organs. Water is lost from the body in routine respiration and via perspiration. Fluid loss of just a few percent can negatively impact cardiovascular function, thermal dissipation, and exercise performance and being in a state of dehydration (or hypohydration) can cause headaches, light-headedness, dizziness, fainting and in extreme cases delirium, unconsciousness or death. Hyponatremia or over-hydration can also detrimentally affect the body's functioning, particularly during exercising, due to electrolyte imbalance.

Dehydration is considered an excessive loss of body fluid. In physiological terms, dehydration may entail a deficiency of fluid within an organism. Dehydration may be caused by losing too much fluid, not drinking enough water or fluids, or both. Vomiting, diarrhea, and excessive perspiration without sufficient liquid intake are other causes of dehydration, which may be particularly worrisome for athletes and people that work under hot and dry conditions. There are three main types of dehydration: hypotonic (primarily a loss of electrolytes, sodium in particular), hypertonic (primarily a loss of water), and isotonic (equal loss of water and electrolytes). While the most commonly seen type of dehydration in humans is isotonic dehydration, distinction of isotonic from hypotonic or hypertonic dehydration may be important when treating people who become dehydrated.

A healthy state of hydration in normal and adverse environments can be maintained when fluid consumption matches fluid loss. Knowledge of an individual's hydration status assists the individual or health care provider in rectifying body fluid imbalances and provides early detection and rapid assessment of thermal or hydration based injury.

Relying upon thirst as a feedback mechanism to trigger demand for fluid intake may not be adequate to maintain an optimal hydration level, since a sensation of thirst sufficient to cause a subject to drink may not be triggered until after the subject is already dehydrated. Adequate hydration is important to maintaining one's health and wellness, there is at present a dearth of non-invasive, rapid devices and methods to analyze data regarding a body fluid analyte collected from a device to ascertain whether a state dehydration, hydration, or euhydration is present.

Some methods to assess hydration status rely on a sample of urine or blood. For example, urine specific gravity is a one approach to ascertain hydration level in the body. Total urine output may be used as a metric. Hydration status can also be assessed using a blood sample, since an increase in plasma osmolality can often identify a state of dehydration, but such sensing requires invasive collection of a venous blood sample by a qualified phlebotomist. For obvious reasons, urine or blood for assessment of hydration status can be highly impractical.

Saliva, however, is an ideal choice for development of a rapid, point-of-care diagnostic measurement for dehydration. The sample is easily obtained with minimal invasiveness. However, saliva is a complex fluid. Approximately 99% of saliva is water, and the remaining 1% consists of large organic molecules such as proteins, small organic molecules such as urea, and electrolytes such as sodium and potassium. Whole saliva, considered as the total fluid content of the mouth, contains many other constituents, including serum components, blood cells, bacteria, bacterial products, epithelial cells, cell products, food debris and bronchial secretions. Saliva is produced by a number of specialized glands that discharge into the oral cavity. Most of the saliva is produced by three pairs of major salivary glands the parotid, submandibular and sublingual glands, and a small amount is made by the numerous small glands that line the mouth.

Methods, systems, devices and analytical algorithms for assessing data from saliva samples, for monitoring dehydration levels or hydration levels in subjects remain a need in the art.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a method to ascertain a hydration level in a human subject is provided. The method comprises determining an indicator concentration value from a saliva sample from a human subject; transforming the indicator concentration value using a variance stabilizing transformation to determine a transformed indicator concentration value; calculating an adjusted indicator concentration value by adjusting the transformed indicator concentration value by a previously determined indicator concentration value for the subject, the previously determined indicator concentration value transformed using a variance stabilizing transformation; and assigning a hydration level to the subject based on the adjusted indicator concentration value, wherein the assigned hydration level is selected from a plurality of levels determined from a statistically treated set of indicator concentration values for a population of human subjects.

In one embodiment, the steps of transforming and calculating comprise using a variance stabilizing transformation selected from the group consisting of a square root function, a logarithmic transformation, and an arc-sin function to provide, respectively, the transformed indicator concentration value and the previously determined indicator concentration value.

In another embodiment, the step of calculating comprises adjusting the transformed indicator concentration value by a previously determined indicator concentration value for the subject that has been logarithmically transformed.

In another embodiment, the logarithmically transformed indicator concentration value is a logarithmically transformed indicator concentration median value, a logarithmically transformed indicator concentration mean value or a logarithmically transformed indicator concentration single value.

In yet another embodiment, when the logarithmically transformed indicator concentration value is a median value or a mean value, it is based on a plurality of indicator concentration values from a single saliva sample from the subject or it is based on an indicator concentration value from a plurality of saliva samples from the subject.

In one embodiment, transforming comprises transforming the indicator concentration value with a logarithmic transformation selected from a log₂, log₁₀, or ln transform.

In another embodiment, transforming comprises calculating an adjusted indicator concentration value by subtracting from the adjusted indicator concentration value a log₂, log₁₀, or ln transformed baseline indicator concentration value for the subject.

In another embodiment, the logarithmically transformed baseline indicator concentration value is a logarithmically transformed median baseline indicator concentration value based on three or more saliva indicator concentration values from the subject.

In still another embodiment, the logarithmically transformed median baseline indicator concentration value is a log₂ transformed median baseline value. In other embodiments, the standard deviation of the three or more log₂ transformed median baseline indicator concentration values is less than about 0.40, or less than about 0.35 or less than about 0.30, or is between about 0.001-0.50 or between about 0.01-0.50 or between about 0.01-0.40, 0.01-0.35.

In one embodiment, the method further comprises adjusting the adjusted saliva indicator concentration value by an exercise adjustment factor if the saliva indicator concentration value is determined from a sample provided while the subject is exercising. In another embodiment, the method comprises adjusting the adjusted saliva indicator concentration value by an elderly-person adjustment factor if the saliva indicator concentration value is determined from a sample provided by an elderly subject. In still another embodiment, the method further comprises adjusting the adjusted saliva indicator concentration value by a medication adjustment factor if the saliva indicator concentration value is determined from a sample provided by a subject on a medication that affects saliva indicator concentration. In yet another embodiment, the method further comprises adjusting the adjusted saliva indicator concentration value by a disease-condition adjustment factor if the saliva indicator concentration value is determined from a sample provided by a person having a disease or condition that affects saliva indicator concentration.

In one embodiment, the adjusting comprises subtracting from the adjusted saliva indicator concentration value a standard deviation (SD) of the statistically treated set of indicator concentration values for the population of human subjects, to provide an exercise adjusted saliva indicator concentration value, and assigning a hydration level based on the exercise adjusted saliva indicator concentration value.

In one embodiment, the SD corresponds to a standard deviation from an analysis of variance of the set of indicator concentration values.

In still another embodiment of the method, the step of assigning comprises assigning a hydration level selected from a plurality of levels determined from a set of indicator concentration values for a population of human subjects, the set of indicator concentration values statistically treated using a statistical tool selected from the group consisting of linear regression analysis, analysis of variance, discriminant analysis and robust regression, and the hydration levels are taken as a standard deviation SD of the statistically treated set of indicator concentration values.

In one embodiment, assigning comprises assigning a dehydration level from a plurality of levels selected from a first hydration level if the adjusted saliva indicator concentration value is less than SD, a second hydration level if the adjusted saliva indicator concentration value is equal to or greater than SD and less than 3 sD, and a third hydration level if the adjusted saliva indicator concentration value is equal to or greater than 3 SD. In other embodiments, the second, third and further hydration levels are more finely defined relative to a calculated SD, for example, a first hydration level is defined to be values less than SD, and a second hydration level is defined to be values greater than SD and less than a·SD, and a third hydration level is defined to be values equal to a SD and greater than b·SD, and a further hydration level is defined to be values equal to b·SD and less than C·SD, and a further hydration level is defined by be values equal to and greater than C·SD, where a, b, c represent a value selected from all values between, and inclusive of the end points, 0.1-10 in increments of one-tenth, 0.1, 0.2, 0.3, 0.4, 0.5 1.0, 1.1, 1.2, 1.3, 1.4, . . . 3.0, 3.1, 3.2, 3.3, 3.4, 3.5, . . . etc.

In another embodiment, SD corresponds to a standard deviation from an analysis of variance of the set of indicator concentration values.

In other embodiments, the step of assigning comprises assigning a hydration level selected from a plurality of levels determined from a statistically treated set of daily indicator concentration values for a population of human subjects.

In still other embodiments, the indicator concentration value is selected from the group consisting of an osmolality value, a conductivity value, a freezing point depression value, a refractive index value. In one embodiment, the indicator concentration value is an osmolality value.

In some embodiments, the saliva sample is a saliva sample from the parotid saliva gland or wherein the saliva sample is a saliva sample from the sub-lingual gland.

In another aspect, a method to determine a hydration level in a human subject is provided. The method comprises determining a saliva osmolality value from a saliva sample from a human subject; transforming the saliva osmolality value to determine a transformed saliva osmolality value; calculating an adjusted saliva osmolality value by adjusting the transformed saliva osmolality value by a previously determined, transformed median baseline osmolality value for the subject; and assigning a hydration level to the subject based on the adjusted saliva osmolality value, wherein the assigned hydration level is based on a standard deviation x of a statistically treated set of osmolality values for the subject, wherein an adjusted saliva osmolality value less than x defines a first hydration level, an adjusted saliva osmolality value equal to or greater than x and less than b·x defines a second hydration level, and an adjusted saliva osmolality value greater than or equal to b·x defines a third hydration level, wherein b corresponds to a value selected from all values between, and inclusive of the end points, 0.1-10 in increments of one-tenth, 0.1, 0.2, 0.3, 0.4, 0.5 1.0, 1.1, 1.2, 1.3, 1.4, . . . 3.0, 3.1, 3.2, 3.3, 3.4, 3.5, . . . etc. In some embodiments, b is 2, 3 or 4. In other embodiments, x corresponds to a standard deviation from an analysis of variance of the set of osmolality values.

In one embodiment, transforming and calculating independently comprise transforming or calculating using a variance stabilizing mathematical transformation. In some embodiments, the variance stabilizing mathematical transformation is a logarithmic transformation. In other embodiments, logarithmically transforming comprises log₂, log₁₀, or ln transforming the saliva osmolality value. For example, in some embodiments calculating logarithmically transforming comprises log₂, log₁₀, or ln transforming the saliva osmolality value.

In still another embodiment, assigning comprises assigning a hydration level based on a set of osmolality values statistically treated using a statistical tool selected from the group consisting of linear regression analysis, analysis of variance, discriminant analysis and robust regression, and the hydration levels are taken as a standard deviation x of the statistically treated set of osmolality values.

In another embodiment, assigning a hydration is based on a statistically treated set of daily indicator concentration values for the subject.

In other embodiments, the method comprises instructing the subject to intake fluid if the hydration level assigned is one that medically correlates to a state of dehydration.

In still other embodiments, the method further comprises repeating the steps of determining, transforming, calculating, assigning, and optionally instructing, at least once within an 8 hour period, or a 10 hour period, or a 12 hour period, or a 2, 3, 4, 5, 6, or 7 hour period.

In other embodiments, the steps of determining, transforming, calculating, and assigning are via a device with a sample receiving region to receive the saliva sample.

In yet another aspect, a method to determine whether a subject is dehydrated is provided, the method comprising determining a saliva osmolality value from a saliva sample; based on the saliva osmolality value, assigning a dehydration level to the subject, wherein the dehydration level is selected from (i) a first dehydration level that corresponds to a threshold value at which greater than about 80% of subjects have an X % or greater body weight loss on a scatter plot of logarithmically transformed saliva osmolality values as a function of percent body weight loss, wherein if the saliva osmolality value is equal to or greater than the threshold value, the first dehydration level is assigned, and (ii) a second dehydration level that corresponds to a saliva osmolality value less than the threshold value.

In one embodiment, assigning comprises assigning a dehydration level selected from dehydration levels wherein the threshold value is determined from a scatter plot of saliva osmolality values transformed by log₂, log₁₀, or ln.

In another embodiment, assigning comprises assigning a dehydration level selected from dehydration levels wherein the threshold value is one where greater than about 80% of subjects have an X % or greater body weight loss, wherein X % is selected from 1%, 2%, 3% or 4%.

In still other embodiments, assigning comprises assigning a dehydration level selected from dehydration levels wherein the threshold value is one where greater than about 90% of subjects have an X % or greater body weight loss on a scatter plot of logarithmically transformed saliva osmolality values as a function of percent body weight loss.

In still another aspect, a method to evaluate hydration level in a subject is provided. The method comprises determining a saliva osmolality value from a saliva sample from a human subject; transforming the saliva osmolality value to a logarithmic value to determine a transformed saliva osmolality value; assigning a hydration level to the subject based on the transformed saliva osmolality value, wherein the hydration level is selected from a plurality of hydration levels determined from a calibration standard curve of logarithmically transformed saliva osmolality values as a function of percent body weight loss.

In one embodiment, assigning comprises assigning a hydration level selected from a plurality of hydration levels determined from a calibration standard curve of saliva osmolality values logarithmically transformed log₂, log₁₀, or ln.

In still another embodiment, transforming comprises log₂, log₁₀, or ln transforming the saliva osmolality value.

In yet another embodiment, body weight loss is measured as reduction in body mass due to water loss. In one embodiment, the initial, first, or starting body mass is obtained when the subject is euhydrated.

In another embodiment, the subject body weight loss is measured in subjects that are euhydrated when an initial saliva sample is obtained and an initial body mass is obtained

Additional embodiments of the present methods will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIG. 1 is a graph of the standard deviation of three saliva osmolality values for individual subjects as a function of the mean of the three osmolality values for each subject;

FIG. 2 shows the steps in the method for assigning a hydration level or dehydration level to a subject, according to one embodiment;

FIG. 3 shows steps conducted by the subject desirous of knowing his/her hydration or dehydration state and the process steps executed by software programmed to execute an algorithm in accord with the methods described herein; and

FIGS. 4A-4B are graphs of a variance stabilized saliva indicator value as a function of percent body weight loss.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

An “indicator” as used herein intends a solute, ion, or analyte in a biological fluid, such as saliva. The amount of indicator present in a biological sample is referred to as the concentration of indicator, or the concentration value of the indicator. It will be appreciated that the amount of indicator in a biological sample can be determined in various ways, depending at least in part on the indicator and on sample. For example, where the indicator is an ion or an electrolyte, conductivity of the sample provides a measure of the amount or concentration of the indicator(s). Where the indicator is a solute, a measure of osmolality (or osmolarity or other measure) corresponds to the amount of indicator in the sample. In one embodiment, the indicator is a indicator of a colligative property of saliva, such as refractive index and specific gravity.

Osmolality is a measure of the osmoles (Osm) of solute per kilogram of solvent (osmol/kg or Osm/kg). Osmolality can be measured, for example, using a freezing point depression osmometer or a vapor pressure depression osmometer. Osmolality is independent of temperature and pressure.

Osmolarity is the number of osmoles of solute per liter (L) of solution (osmol/L or Osm/L). For a given solution, osmolarity is slightly less than osmolality, because the total solution weight (the divisor used for osmolality) excludes the weight of any solutes, whereas the total solution volume (used for osmolarity) includes solute content.

As used in this specification, the singular forms “a,” “an,” and “the” include plural referents unless the context clearly dictates otherwise.

### II. Method for Determining Hydration Level or Dehydration Level

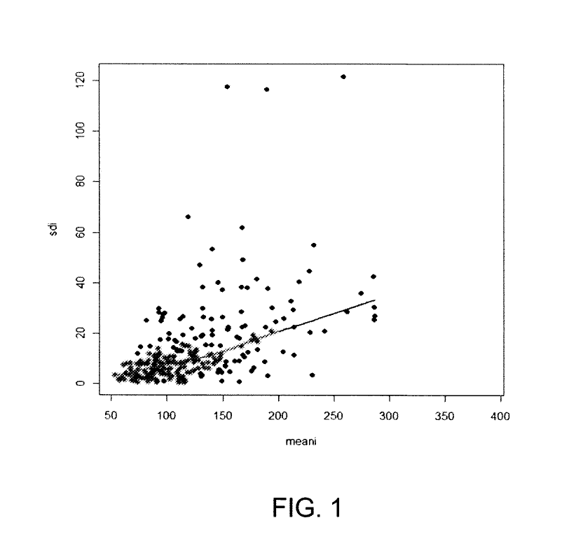

In a first aspect, a method for ascertaining a hydration level in a human subject is provided. The method comprises determining amount or concentration of an indicator in a saliva sample from the subject and then mathematically transforming the saliva indicator concentration value with a variance stabilizing transformation. This transformed value is then adjusted by a previously determined ‘baseline’ indicator concentration value to assign a hydration level to the subject. This method will now be described with reference to Example 1 and FIG. 1.

As detailed in Example, in a first study at least three saliva samples were collected from hydrated human subjects. In the study, osmolality was used as the measure of indicator (solute) concentration in the saliva, although it will be appreciated that other indicators and measures appropriate for that indicator are contemplated and suitable, such as conductivity for a measure of the amount of ion or electrolyte as the indicator, refractive index specific gravity, viscosity, and freezing point depression, as measures of amount of indicators (solutes, ions, analytes) in a sample. Although the working examples herein use osmolality of a saliva sample as an indicator of hydration/dehydration, the method is not intended to be so limited in view of the alternative, suitable indicators, and methods know to assess amount of one or more indicators, such as those just mentioned. The indicator amount in the saliva samples as determined by an osmolality value was determined for each of the three hundred saliva samples, where identification of each sample was tracked to determine a mean osmolality baseline value for each subject and a standard deviation in osmolality for each subject. A plot of subject standard deviation as a function of subject mean baseline osmolality value for each subject is shown in FIG. 1.

FIG. 1 shows that the variation in each subject's standard deviation in saliva indicator value (osmolality in this embodiment) increases as a linear function of the mean indicator value for each subject, which is indicative of a log-normal distribution. The method, therefore, comprises transforming the indicator value, such as an osmolality value, with a variance stabilizing mathematical transform to yield a transformed indicator value. The method, therefore, also comprises applying a variance stabilizing transformation to a previously determined indicator concentration value, and adjusting the transformed indicator concentration value by the variance stabilized, previously determined indicator concentration value to yield an adjusted indicator concentration value. The variance stabilizing mathematical transform applied to the indicator values can be any one of the variance stabilizing transformations known to those skilled in the art. Exemplary transformations include the Fischer transformation, a square root transformation, the arcsine square root transformation, logarithamic transformation, and angular transformation. A logarithmic transformation of indicator concentration values intends any log transformation, including but not limited to natural log(ln), base 10 log(log₁₀), base 2 log(log₂), and the like.

With respect to the data presented in FIG. 1, the osmolality values were log₂ transformed and each subject's individual baseline, or ‘previously determined’, indicator concentration value, was taken as the median osmolality value from a plurality of log₂ transformed osmolality values. In one embodiment, the previously determined, indicator concentration value is determined from at least three, at least four, or at least five salivary indicator concentration values from the subject. In another embodiment, the previously determined, indicator concentration value is determined from three or more, five or more, seven or more, nine or more salivary indicator concentration values from the subject. It will be appreciated that the indicator concentration values can be determined from separate, distinct saliva samples from the subject over a period of time (e.g., several hours, a day, several days, a week, etc.) or can be determined from repeated measurements of indicator concentration from one saliva sample or from a small number (e.g., 2-3, 3-5, or 2-5) saliva samples from the subject.

Accordingly, in the method for determining a hydration level or dehydration level, an indicator concentration value from the saliva of a subject is determined. The indicator concentration value is transformed with a variance stabilizing mathematical transformation to yield a transformed indicator concentration value. For the subject, a previously determined indicator concentration value is known, wherein the previously determined indicator concentration value has been treated with a variance stabilizing transformation. The transformed indicator concentration value is adjusted by the previously determined, variance stabilized indicator concentration value to provide an adjusted indicator concentration value. An exemplary adjusted indicator concentration value, Δ, is equal to the saliva indicator concentration value, x, that is log transformed minus the previously determined, baseline, indicator concentration value, BV: Δ=log(χ)−(BV). Based on the adjusted indicator concentration value Δ, a hydration level (or dehydration level) is assigned to the subject, where the hydration levels are determined from a set of indicator concentration values for a population of subjects, as will now be described.

Using the data from the study in Example 1, an analysis of variance (ANOVA) was conducted on the within-subject values, including replicates at each time point and measurements taken at various times during the day. The ANOVA found the replicate standard deviation (SD) was 0.12, and the daily standard deviation was 0.27, which corresponds to approximately 20%. Using the daily standard deviation, a plurality of hydration levels or ‘categories’ were defined, wherein a ‘normal’ hydration level was defined as values within 1 SD, and additional dehydration categories 1-2 SD's, 2-3 SD's, and >3 SD's. This approach was then prospectively validated in data from a second study (referred to herein as Study 2), as will now be described.

To test the assumption in the method that a plurality of hydration levels for any individual subject can be determined from the standard deviation of indicator concentration values from a population of subjects, the effect of demographic factors on the indicator concentration value variation was evaluated empirically. If the individual demographic factors are significantly associated with baseline variation, the assumption, and thus the methodology, would not be valid for all subjects, and instead a ‘subject specific threshold’ indicator concentration value would be needed. Data from two different studies, each with a sample size of at least 100 subjects, was used. In Study 2, 109 subjects were included and 649 saliva samples were collected. Demographic data on each subject was obtained. For each subject, multiple saliva samples were collected, and a within subject standard deviation (SD) was calculated for each subject. The within subject standard deviation was then analyzed using analysis of variance (ANOVA), which assessed possible contributions to the within subject standard deviation from the following sources: age, gender, weight, tobacco use, and activity level. The results are shown in Table 1.

TABLE 1
Analysis of Variance on Demographic Data from Subjects in Study 2

| Demographic | | Standard | | |
|---|---|---|---|---|
| Factor | Estimate | Error | T statistic | p-value |
| Age (yrs) | 0.0005 | 0.0008 | 0.66 | 0.51 |
| Gender | 0.027 | 0.023 | 1.18 | 0.24 |
| (Male) | | | | |
| Weight (lbs) | 0.0003 | 0.0003 | 1.09 | 0.28 |
| Tobacco Use | 0.006 | 0.025 | 0.25 | 0.80 |
| Activity Level | −0.004 | 0.009 | −0.42 | 0.68 |

As seen in Table 1, none of the demographic factors studied were found to have a significant effect (e.g., p<0.05) on baseline osmolality (indicator value) variation.

A similar analysis was conducted on the data collected from the 100 subjects in Study 1 of Example 1. The results of the ANOVA are shown in Table 2.

TABLE 2
Analysis of Variance on Demographic Data from
Subjects in Study 1 in Example 1

| Demographic | | Standard | | |
|---|---|---|---|---|
| Factor | Estimate | Error | T statistic | p-value |
| Age (yrs) | 0.0005 | 0.003 | 0.20 | 0.85 |
| Gender | 0.036 | 0.053 | 0.69 | 0.49 |
| (Male) | | | | |
| Weight (lbs) | 0.0005 | 0.0008 | 0.66 | 0.51 |

As in the data from Study 2 (Table 1), the analysis of the data from the subjects in Study 1 described in Example 1 found that osmolality variation was not related to common demographic factors. Together, these results support the validity of the approach of using the hydration (or dehydration) levels for any individual subject based on the standard deviation of indicator concentration values from a population of subjects. In one embodiment, the plurality of hydration (or dehydration) levels are obtained by measuring an indicator concentration value for a population of subjects, wherein the population includes at least 10 subjects, at least 25 subjects, at least 50 subjects, at least 100 subjects or at least 200 subjects. The indicator concentration value for each subject in the population is determined from a saliva sample, wherein in various embodiments the saliva sample is taken once per day, twice per day, or three times per day. In some embodiments, a single saliva sample can be measured more than once to obtain more than one indicator concentration value. The indicator concentration values, preferably for each saliva sample at different time points from each subject, are analyzed statistically to determine a plurality of hydration levels. For example, the data set comprised of indicator concentration values for a population of subjects can be analyzed or manipulated with a variety of statistical tools, such as analysis of variance, linear regression, discriminant analysis and robust regression, for example. Other statistical treatments will be apparent to one skilled in the art. Hydration levels are selected based on a standard deviation value, SD, of the statistically-treated set of indicator concentration values. By way of example, and as mentioned above, in one embodiment an analysis of variance was performed on the osmolality data obtained from daily saliva samples from the subjects in Study 1. The standard deviation, SD, of the ANOVA-treated data was determined. Using this value SD, hydration/dehydration levels can be determined for any adjusted indicator concentration value A, for example, as follows:

| | | |
|---|---|---|
| LEVEL 1: | NORMAL HYDRATION: | Δ < SD |
| LEVEL 2: | MODERATELY DEHYDRATED: | SD ≦ Δ < 2SD |
| LEVEL 3: | DEHYDRATED: | 2SD ≦ Δ < 3SD |
| LEVEL 4: | SEVERELY DEHYDRATED: | 3SD ≦ Δ |

It will be appreciated that the number of levels in the plurality of levels and the threshold or cut-off values to move between levels can vary, and the four levels noted above are merely exemplary. In other embodiments, the plurality of hydration/dehydration levels corresponds to three levels; in other embodiments, a level indicating that the subject is euhydrated is included in the plurality of levels (e.g., Δ<0.5 SD); in other embodiments, the plurality of levels or categories is comprised of two levels, a first level or category to indicate hydration is normal and a second level or category to indicate that an intake of fluid is recommended. In another embodiment, the cut-off value or SD value that delimits each level is approximately 10%, 15%, 20%, 25%, 50% or 75% of the standard deviation in a statistically treated (e.g. ANOVA-treated) data set of indicator concentration value (e.g., osmolality) values from a population of subjects. In another embodiment, the cut-off value or SD value that delimits each level is a value greater than a standard deviation in a statistically treated (e.g. ANOVA-treated) data set of indicator concentration value (e.g., osmolality) values from a population of subjects, for example, a value 1.5 times, 2 times, 2.5 times or 3 times the standard deviation.

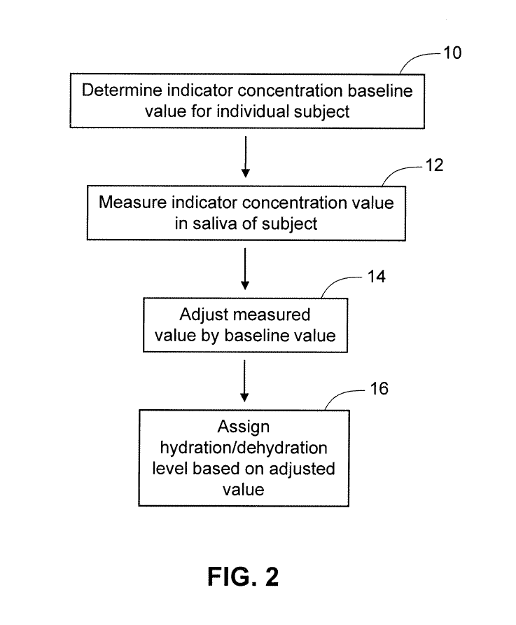

FIG. 2 shows the basic steps in the method for assigning a hydration level or dehydration level to a subject. A baseline value of an indicator concentration in the saliva of the subject is determined, 10, for example, by the subject measuring saliva osmolality (or specific gravity, or conductivity, or refractive index, etc.) to obtain, in a preferred embodiment, at least three values. The values can be obtained once per day, or more or less frequently. Once the subject's baseline indicator concentration value is established, the subject then measures his/her saliva indicator concentration value at any time s/he wishes to know his/her hydration level, 12, using a device such as that described herein below. The device is programmed to calculate an adjusted indicator concentration value by adjusting the measured indicator concentration value by the baseline indicator concentration value, 14. The adjusted indicator concentration value is then used to assign a hydration/dehydration level, 16, according to the levels determined from a statistically treated data set of indicator concentration values from a population of subjects.

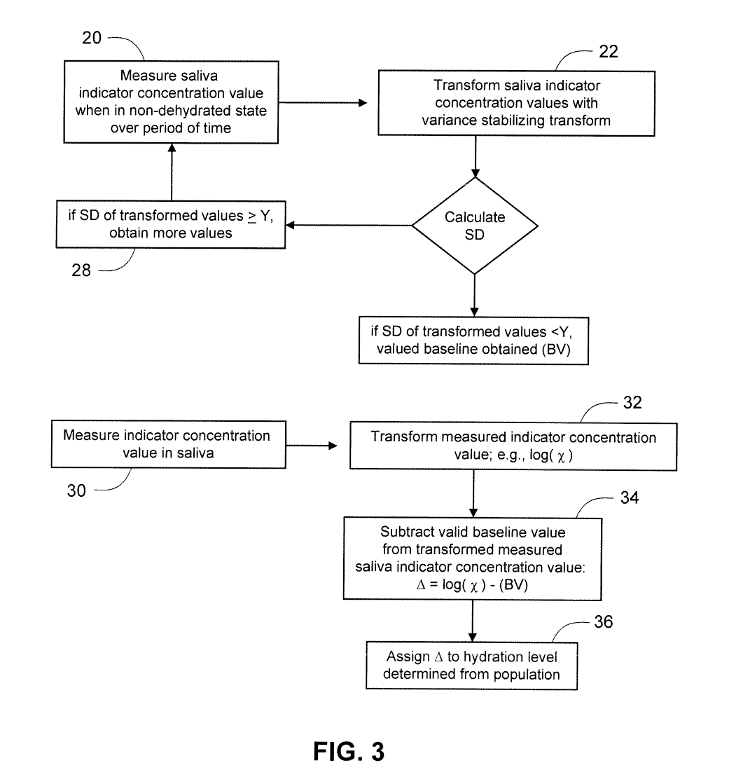

A more detail flow is illustrated in FIG. 3. A subject desirous of knowing his/her hydration or dehydration state obtains a plurality of indicator concentration values from one or more saliva samples, preferably when the subject has ensured s/he is fully hydrated, for example, by intaking plenty of fluid over the prior 12-24 hours, 20. The indicator concentration values are statistically treated using a variance stabilizing transformation, such as a logarithmic transform, 22, for example, a log₂, log₁₀ or logₑ (ln) transform. In one embodiment, the plurality of indicator concentration values that are statistically treated using a variance stabilizing transformation, for ascertaining the ‘baseline’ or ‘previously-determined indicator concentration value’ for use in the algorithm, comprises at least two, or at least three, or at least four or at least five values. The standard deviation, SD, of the variance stabilized plurality of indicator concentration values is determined, 24. If the standard deviation of the variance-stabilized plurality of indicator concentration values is less than a selected value, Y, then the baseline (also referred to herein as the previously-determined indicator concentration value) is valid, 26. If the standard deviation of the variance-stabilized plurality of indicator concentration values is equal to or greater than the selected value, Y, then additional indicator concentration values should be obtained and added to the plurality of values to determine a valid baseline, By, for the subject, 28.

Once the baseline for the subject is established, the hydration or dehydration state can be ascertained. The subject measures a indicator concentration value, x, of his/her saliva, 30. The indicator concentration value is transformed with a variance stabilizing mathematical transformation to yield a transformed indicator concentration value, log(χ) 32. The previously-determined baseline value, BV, is subtracted from the transformed indicator concentration value, for example if a logarithmic transformation is used log(χ), to yield an adjusted indicator concentration value, Δ, 34. Then, based on the value Δ, a hydration/dehydration level is assigned, 36, the levels determined from a statistically-treated data set of indicator concentration values from a population of subjects, as discussed above.

The algorithm described above was validated with regard to its specificity analysis using data sets on several studies where saliva samples were obtained at various time points in conjunction with a measure of body weight. More specifically, three studies were conducted wherein saliva samples from subjects were obtained. Information on the studies are set forth in Table 3.

TABLE 3
Details of Study 3, Study 4 and Study 5

| | | | Conditions |
|---|---|---|---|
| | | No. of Saliva | during |
| | No. of | Samples for | sample |
| Study No. | Subjects | Analysis | collection |
| Study 3 | 8 | 134 | exercising |
| Study 4 | 12 | 312 | exercising |
| Study 5 | 23 | 254 | exercising |

Subjects in Study 3, Study 4 or Study 5 wherein no change in body weight occurred were included in the specificity analysis. With this criterion applied, 129 saliva samples were associated with a confirmed zero percent body weight loss, and these saliva samples were used as baseline samples. Indicator concentration values in the form of osmolality values were obtained from the 129 baseline saliva samples, and the values were treated according to the algorithm described above. A baseline indicator concentration value for each individual was established, and hydration levels were determined based on the collective data set of indicator concentration values in the population of saliva samples. The values were transformed and statistically-treated as described. Analysis of the results in shown in Table 4.

TABLE 4
Specificity Analysis of Algorithm based on data from subjects in
Study 3, Study 4 and Study 5 with no body weight loss

| | No. of | | “Moderately | | “Very |
|---|---|---|---|---|---|
| % Body | baseline | “Normal” | Elevated” | “Elevated” | Elevated” |
| Weight | saliva | Hydration | Dehydration | Dehydration | Dehydration |
| Loss | samples | Level | Level | Level | Level |
| 0 | 129 | 122 | 6 | 1 | 0 |

| | | | | |
|---|---|---|---|---|
| Statistical | 95% | 4.7% (6/129) | 0.8% (1/129) | 0% |
| Analysis of data | (122/129) | | | |

The data in Table 4 shows that the specificity ranges from 95-99%, depending on whether the moderately elevated category is deemed to be dehydrated.

An aggregated analysis was performed on subjects from all studies with baseline samples taken under normal, non-exercising, conditions. In contrast to the specificity analysis samples, these samples are from subjects not confirmed to have been normally hydrated; rather, these samples constitute an assessment of normal variation. This analysis includes samples from an additional study, Study 6, where 33 subjects were enrolled and 315 saliva samples were obtained with the subjects in a normal, non-exercising condition. In this aggregated analysis, the samples from Study 2 were also included. The saliva samples from Study 1 (Example 1) were not included in this assessment since they were used to develop the algorithm.

TABLE 5
Analysis of Algorithm based on data from subjects in
Study 3, Study 4, Study 5 and Study 6

| | | | “Moderately | “Elevated” | “Very |
|---|---|---|---|---|---|
| % Body | No. of | “Normal” | Elevated” | Dehy- | Elevated” |
| Weight | saliva | Hydration | Dehydration | dration | Dehydration |
| Loss | samples | Level | Level | Level | Level |
| Baseline | 964 | 861 | 80 | 22 | 1 |

| | | | | |
|---|---|---|---|---|
| Statistical Analysis | 89% | 8.3% | 2.3% | 0.1% |
| of data | | | | |

As the data in Table 5 indicates, under normal (uncontrolled) conditions, approximately 90% of the salvia samples are in the normal range indicating the subject is hydrated, 8% in the moderately elevated range, indicating a moderate amount of dehydration, and 2% in the elevated range indicating dehydration. After removal of subjects who didn't meet the acceptable baseline requirements, the normal variation distribution is shown below. As expected, applying the algorithm baseline flag removed some of the more extreme values from the normal variation distribution.

TABLE 6
Analysis of Algorithm

| | | | “Moderately | “Elevated” | “Very |
|---|---|---|---|---|---|
| % Body | No. of | “Normal” | Elevated” | Dehy- | Elevated” |
| Weight | saliva | Hydration | Dehydration | dration | Dehydration |
| Loss | samples | Level | Level | Level | Level |
| Baseline | 928 | 840 | 71 | 15 | 0 |

| | | | | |
|---|---|---|---|---|
| Statistical Analysis | 91% | 7.7% | 1.6% | 0% |
| of data | | | | |

The algorithm (method) described herein was also validated for sensitivity. In this validation, data from three studies that induced dehydration, quantified using percent body weight loss (BWL) as a measure, were Study 3, Study 4 and Study 5. A combined analysis of algorithm sensitivity as a function of % BWL is shown below in Table 7. For values of % BWL greater than 3%, the algorithm identified 100% of subjects at least moderately elevated, 96% elevated or greater, and 67/75=89% very elevated. At a % BWL of 3%, the algorithm identified 98% of subjects at least moderately elevated. For lower values of % BWL (1-2%), subjects tended to be spread across the dehydration/hydration categories. The rate of “very elevated” algorithm calls in this range of % BWL suggested that an adjustment factor would be beneficial to avoid overcalling some subjects undergoing vigorous exercise who were not yet very dehydrated.

TABLE 7
Analysis of Algorithm

| | | | “Moderately | | “Very | | |
|---|---|---|---|---|---|---|---|
| % Body | No. of | “Normal” | Elevated” | “Elevated” | Elevated” | | |
| Weight | saliva | Hydration | Dehydration | Dehydration | Dehydration | Sens | Sens |
| Loss | samples | Level | Level | Level | Level | (Mod+) | (Elev+) |
| 1 | 106 | 59 | 25 | 12 | 10 | 44% | 21% |
| 2 | 61 | 12 | 14 | 13 | 22 | 80% | 57% |
| 3 | 58 | 1 | 8 | 13 | 36 | 98% | 84% |
| 4 | 29 | 0 | 3 | 2 | 24 | 100% | 90% |
| 5 | 18 | 0 | 0 | 2 | 16 | 100% | 100% |
| 6+ | 28 | 0 | 0 | 1 | 27 | 100% | 100% |

Based on the findings in the data shown in Table 7, a similar sensitivity analysis was performed using an exercise adjustment to the algorithm. The algorithm continued to have good sensitivity for % BWL>2% (91-100%) while reducing the sensitivity to smaller values of % BWL, as seen in the data in Table 8.

TABLE 8
Analysis of Algorithm with Exercise Adjustment Factor

| | | | “Moderately | | “Very | | |
|---|---|---|---|---|---|---|---|
| % Body | No. of | “Normal” | Elevated” | “Elevated” | Elevated” | | |
| Weight | saliva | Hydration | Dehydration | Dehydration | Dehydration | Sens | Sens |
| Loss | samples | Level | Level | Level | Level | (Mod+) | (Elev+) |
| 1 | 106 | 77 | 16 | 10 | 3 | 27% | 12% |
| 2 | 61 | 25 | 13 | 10 | 13 | 59% | 38% |
| 3 | 58 | 5 | 14 | 16 | 23 | 91% | 67% |
| 4 | 29 | 0 | 4 | 5 | 20 | 100% | 86% |
| 5 | 18 | 0 | 1 | 5 | 12 | 100% | 94% |
| 6+ | 28 | 0 | 0 | 3 | 25 | 100% | 100% |

In summary, in the prospectively defined analysis of multiple studies, the algorithm showed high specificity (95%) and high sensitivity (91-100%) for % BWL greater than 2%. The analysis also indicted that for some subjects, an adjustment factor to the algorithm is desirable. Accordingly, in one embodiment, the method includes adjusting the indicator concentration value by an adjustment factor. In one embodiment, the adjustment factor is an exercise adjustment factor if the saliva sample or the saliva indicator concentration value is provided or obtained while the subject is exercising or just after the subject exercised. The method contemplates other adjustment factors, such as an elderly-person adjustment factor if the saliva indicator concentration value is determined from a saliva sample provided by an elderly person, which is one embodiment intends a person that is 65 years of age or older, or is 70 years of age or older, or is 75 years of age or older. A medication adjustment factor can be utilized if the saliva sample is obtained from a subject on a medication that affects saliva indicator concentration values, such as a diuretic, steroids, beta blockers, antidepressants and stimulants. A disease-condition adjustment factor can also be applied, if the saliva sample is from a person with a disease or condition that affects saliva indicator concentration values (relative to a non-medicated population). For example, subjects with conditions which cause an abnormal secretion of anti-diuretic hormone are candidates for an adjustment factor in the algorithm to adjust for changes in saliva indicator concentration values due to the condition. Likewise, individuals with congestive heart disease, renal disease, Crohn's disease, diabetes and autoimmune diseases may have a normally higher or lower baseline and benefit from an adjustment factor in the algorithm.

Also contemplated herein is a method to determine whether a subject is dehydrated wherein a baseline, or previously-determined, indicator concentration value is not required. In this embodiment, a saliva indicator value is determined for the subject and a dehydration level is assigned based on a previously established correlation of, for example, indictor value and percent body weight loss. In one embodiment, the dehydration level is selected from (i) a first dehydration level that corresponds to a threshold value at which greater than about 80% of subjects have an X % or greater body weight loss on a scatter plot of variance stabilized transformed saliva indicator values as a function of percent body weight loss, wherein if the saliva indicator value is equal to or greater than the threshold value, the first dehydration level is assigned, and (ii) a second dehydration level that corresponds to a saliva indicator value less than the threshold value. In one embodiment, the variance stabilized transformed saliva indicator value is a logarithmically transformed indicator concentration value. In one embodiment, X % corresponds to at least about 3%, 2% or 1%.

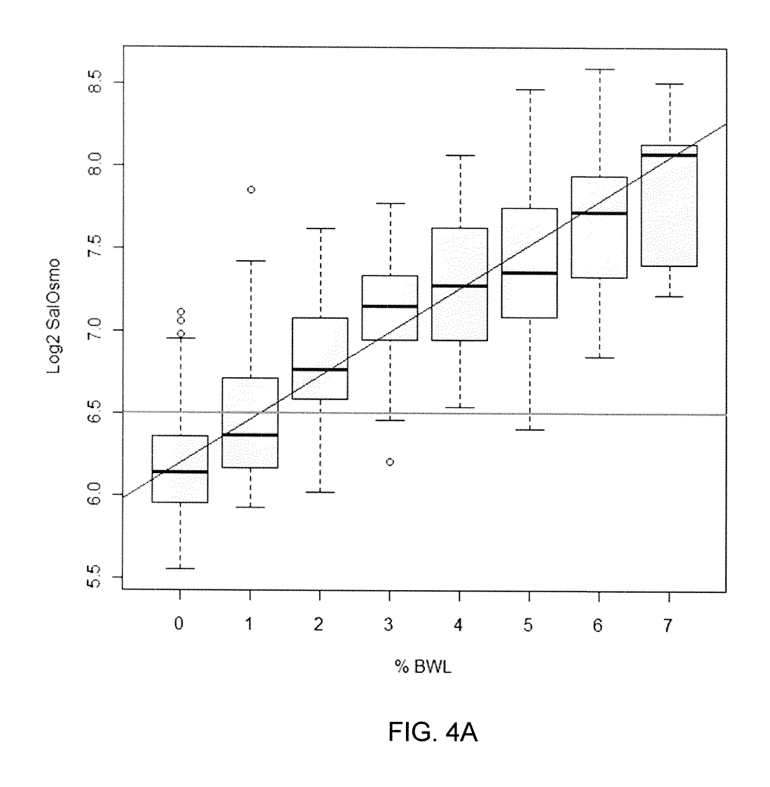

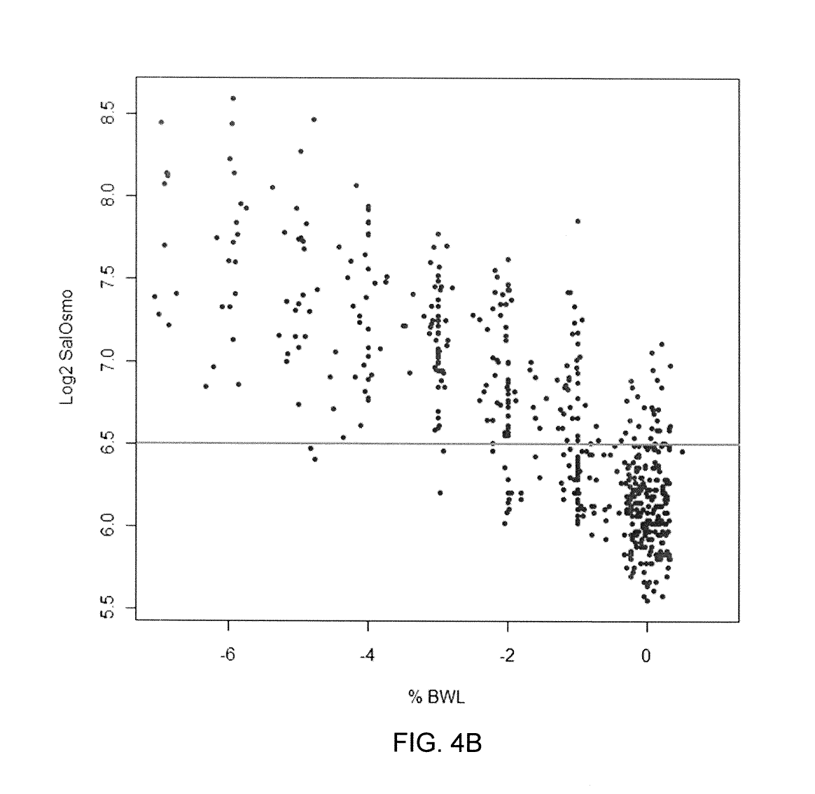

The validity of this method is shown with respect to the data from the six studies described above. The combined number of saliva samples for analysis from the six studies was 1882. The osmolality of the 1882 saliva samples was determined, as the exemplary indicator value for the method. The osmolality values were transformed with a variance stabilizing transformation, which, in this example, was a logarithmic (log 2) transformation. The transformed values were associated with percent body weight loss for the subject. The data was inspected to identify the threshold at which 95% of the subjects with 2% or higher body weight loss occur. A graph was constructed of transformed osmolality values as a function of percent body weight loss, as shown in FIGS. 4A-4B. The threshold at which 95% of the subjects who had 2% body weight loss or more was 6.5 on the log 2 scale, which corresponds to about 90 mOsmo. At this threshold, shown by the solid line in each figure, 12.6% of non-dehydrated samples are above the threshold and 87.4% are below the threshold.

Accordingly, the method of this embodiment comprises, in one embodiment, assigning a dehydration level selected from dehydration levels wherein the threshold value is determined from a scatter plot of saliva osmolality values transformed by a logarithmic transformation, such as log₂, log₁₀, or ln. Assigning a dehydration level, in another embodiment, comprises assigning a level selected from dehydration levels wherein the threshold value is one where greater than about 80% of subjects have an X % or greater body weight loss, wherein X % is selected from 1%, 1.5%, 2%, 2.5%, 3%, 3.5%, 4%, 4.5%, 5%, 5.5%, 6%, 6.5% or 7%.

In another embodiment, assigning comprises assigning a dehydration level selected from dehydration levels wherein the threshold value is one where greater than about 90% of subjects have an X % or greater body weight loss on a scatter plot of logarithmically transformed saliva osmolality values as a function of percent body weight loss.

It will be appreciated that the dehydration levels in this embodiment include at least one level indicative of the subject in a non-dehydrated state, i.e., hydrated. Accordingly, reference to a ‘dehydration level’ can include a level indicating a state of hydration or non-dehydration.

In another embodiment, a method to evaluate hydration level in a subject is provided. In this method, a saliva indictor value from a saliva sample from a human subject is determined, in accord with the indicators and measures described above. The saliva indicator value is transformed using a variance stabilizing transformation, for example, by transforming the value to a logarithmic value to determine a transformed saliva indicator value. Then, a hydration level to the subject is assigned, based on the transformed saliva indicator value. The hydration level is selected from a plurality of hydration levels determined from a calibration standard curve of variance stabilized, transformed saliva indicator values as a function of percent body weight loss. The data in FIGS. 4A-4B are exemplary of this method, wherein a hydration level is assigned from a plurality of hydration levels determined from a calibration standard curve of saliva osmolality values logarithmically transformed log₂, log₁₀, or ln.

In one embodiment, body weight loss is measured as reduction in body mass due to water loss. The subject body weight loss can be measured in subjects that are euhydrated when saliva samples are obtained for determining saliva osmolality values.

A. Devices and Methods to Measure Saliva Indicator Concentration

In one embodiment, the methods described herein are in the form of software code stored on or loadable into a memory device or hardware of a hydration monitoring device. The software code is programmed to execute an algorithm in accord with the methods described herein. Exemplary devices are now described.

In one embodiment, a microcantilever device with a polymeric sensing material responsive to osmolality is contemplated, for use by a subject in measuring a salivary osmolality value as the indicator in saliva to inform regarding hydration/dehydration level. Exemplary microcantilever devices are described, for example, in U.S. Pat. Nos. 6,523,392; 7,168,294; 7,395,693; 7,726,175 and U.S. Published Patent Application No. 20050164299, each of which is hereby incorporated by reference in its entirety.

In certain embodiments, an apparatus for sensing state of euhydration, hydration or dehydration is arranged to generate a user-perceptible output signal indicative of hydration status that is quantitative in character. A user perceptible output signal generated by such an apparatus may be visible, audible, and/or tactile in character. Examples of quantitative signals include salivary conductivity, osmolality, and/or concentration of any indicator in saliva. In certain embodiments, an apparatus for sensing state of euhydration, hydration or dehydration is arranged to generate a user-perceptible output signal indicative of hydration status that is qualitative in character. With respect to indicator concentration values or baseline values or threshold values, for an indicator concentration indicative of euhydration, hydration or dehydration, these values may be stored in memory of the apparatus, and sensed values may be compared to the one or more threshold values to provide a simple qualitative assessment as to whether a user is in a state of euhydration, hydration or dehydration, wherein the apparatus includes software code that executes one or more of the methods described herein.

### Saliva Collection

As noted above, saliva is produced by three pairs of large glands (parotid, submandibular and sublingual) and many smaller glands found throughout the oral mucosa. The saliva from each of these sources varies in composition, viscosity and quantity. Many factors, including stimulation and flow rate, have a marked effect on salivary composition. The parotid gland saliva is consistently and virtually pure serous, and submandibular saliva and sublingual gland saliva is a mixture of mucous and serous saliva.

In one embodiment, the saliva sample used in the method described herein is sublingual saliva or whole saliva, the latter intending fluid produced by the major and minor salivary glands. In one embodiment, the saliva sample is not a saliva sample from the parotid gland. The method comprises, in some embodiments, a step of collecting, providing or receiving a saliva sample, wherein the saliva sample is a sublingual saliva sample or a whole saliva. In other embodiments, collecting or collection of a saliva sample contemplates physically collecting a saliva sample such that the saliva is no longer within the oral cavity, but is, for example, in a container, vial, or device that receives the sample. In other embodiments, collecting or collection of a saliva sample contemplates a sample of saliva that is in the oral cavity, such as by inserting a device that measures an indicator concentration value into the mouth, such as those described above. Accordingly, in some embodiments, a device that can be inserted into a patient's oral cavity is provided, the device having a sensing means to ascertain the indicator concentration value in the saliva, and the software for conducing the method described herein.

In other embodiments wherein a saliva sample is collected into a container or vial or device outside of the oral cavity, the salvia is collected for a collection period of at least about 30 seconds, more preferably of at least about 60 seconds. Use of a salivary stimulating agent is contemplated, and it can applied to a subject prior to or concurrent with placement of a liquid collection element (or portion thereof) in the subject's mouth. The salivary stimulating agent may be arranged for gustatory and/or olfactory stimulation of saliva production. Examples of gustatory salivary stimulating agents include (but are not limited to) citric acid, sodium chloride, or sugarless sour candy. Mechanical stimulation of saliva production (e.g., a mechanical salivary stimulating element) may also be used. In certain embodiments, a chewable article such as chewing gum is administered to a user prior to or concurrent with insertion of a device or placement of a liquid collection element (or portion thereof) in a subject to mouth, in order to stimulate saliva production by chewing.

B. Exemplary Patient Populations

Adequate hydration is a requirement for every human, and the methods described herein are contemplated for use in all humans. In various embodiments, certain patient populations benefit from hydration monitoring, and exemplary patient populations are now described.

In one embodiment, the subject is an exercising subject. The subject may be engaged in any type of physical exercise, and may be a professional athlete or not. In another embodiment, the subject is one undergoing or scheduled to undergo a medical procedure wherein proper hydration is beneficial, such as to reduce the probability of an adverse event. For example, it has been observed that dehydration increases the risk of radiocontrast nephropathy (RCN) when radiocontrast agents are injected into a patient during coronary and peripheral vascular catheterization procedures.

In another embodiment, the subject is one undergoing hemodialysis. Maintaining hemostasis during hemodialysis is recommended to minimize cardiovascular and other associated risks. Edema (suffering from vascular insufficiency of a limb) is difficult to detect until the interstitial fluid volume has risen to approximately 30% above normal, whilst severe dehydration can develop before the onset of clinical symptoms. The current methods can be used to evaluate hydration status of a dialysis patient.

In another embodiment, the patient is an infant or child under 12 years for age. Infants and children are easily dehydrated with possible severe outcomes, and a rapid, simple measure of hydration level is easily provided using the methods described herein.

In summary, the present invention is directed to a non-invasive method of determining hydration levels of an individual. It is important to note that the present invention is not limited to the detection of hypohydration or hyperhydration, but to the rapid and continuous monitoring of body hydration levels to detect for hydration imbalance. The methods of the present invention may utilize a miniature microfluidic salivary monitor to determine the hydration levels in the user without interfering with speech, respiration and deglutition. The present methods utilize an indicator or biomarker in saliva to determine hydration levels. This method is particularly effective in determining hydration levels in athletes, first responders, armed forces, combat casualty situations and for others users where the risk of hydration imbalance is prevalent. Early detection and intervention by health care providers provides necessary hydration therapy without resulting in serious injuries or the unnecessary loss of life. As stated above, the method of the present invention measures at least one salivary component or indicator to determine hydration. The “salivary component” or salivary indicator refers to the physical components of saliva, such as but are not limited to, salivary osmolality, salivary osmolarity, salivary amylase and total salivary protein, salivary conductivity, etc. In one embodiment, the osmolality of pure parotid saliva is used in the method.

In another embodiment, the patient is an elderly person. The thirst reflex is diminished with age, resulting in a substantial fraction of elderly people in a chronic state of dehydration. Some conditions common in the elderly population, such as prostate disease in men or incontinence in women, can result in debilitating dehydration in order to avoid embarrassing or uncomfortable situations. An indicator of hydration for self-assessment or care-giver assessment is beneficial to managing health and avoiding hydration-related emergencies in elderly persons.

### III. Examples

The following examples are illustrative in nature and are in no way intended to be limiting.

### Example 1

### Determining a Baseline Value from Saliva Samples for Dehydration Analysis

One-hundred human subjects aged 65 and older were enrolled in a study, referred to herein as Study 1. Of the 100 subjects, 50 were resident in supervised skilled nursing facilities, and 50 were not. For each subject, a thorough medical history, including past and current medical conditions and current medications, was taken.

The enrolled subjects met with medical staff on 3 days in a 7 day window. Urine, serum and saliva sample collection occurred on visit days. These were Day 1, Day 3 and Day 7. Day 1 and Day 3 were intended as subject normal assessment, and Day 7 was intended to be a slightly more hydrated state. Subjects were not given any special instructions about fluid consumption prior to Day 1 and Day 3 visits. However after Day 3 visit, subjects were asked to drink 3 of 8 oz glasses of water per day for the following 3 days. Final samples were collected on Day 7.

Whole non-stimulated saliva was collected by the following method: subjects were asked to clear existing saliva by swallowing once and then were asked to passively allow saliva to pool in their mouth for two minutes. At the two minute mark, the subjects were asked to transfer the saliva to a clean vial by spitting through a short straw. Each collection event consisted of 3 samples collected in this manner over a 15 minute period. Each sample was labeled and stored in a cooler pack until it could be measured on the freezing point osmometer (Model Fiske 210) later that day. Three saliva samples each were collected on 3 separate days, with a maximum possible number of samples per subject being 9. Sample collection was timed such that no food or beverage had been consumed in the last 60 minutes.

Using the freezing point osmometer, osmolality as the indicator concentration value of the 300 samples was measured. The identification of the samples was tracked to determine a mean osmolality baseline value for each subject and a standard deviation for each subject. A plot of subject standard deviation as a function of subject mean baseline osmolality value for each subject is shown in FIG. 1.

Next, the osmolality values were log₂ transformed, and the median on the log₂ scale was used to estimate each subject's individual baseline osmolality value.

An analysis of variance was performed on the within-subject osmolality values, including replicates at each time point and measurements taken at various times during the day. The analysis of variance identified the replicate standard deviation of 0.12 and a daily standard deviation of 0.27, which corresponds to approximately 20%. Using the daily standard deviation (SD), four hydration categories were defined such that ‘normal’ hydration was defined as baseline adjusted osmolality values within (±)1 SD, moderately dehydrated as values within (±) 1-2 SD's, dehydrated as values within (±) 2-3 SD'S, and severely dehydrated as values >3 SD's.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A method to ascertain a hydration level in a human subject, comprising:
determining an indicator concentration value from a saliva sample from a human subject;
transforming the indicator concentration value using a variance stabilizing transformation to determine a transformed indicator concentration value;
calculating an adjusted indicator concentration value by adjusting the transformed indicator concentration value by a previously determined indicator concentration value for the subject, the previously determined indicator concentration value transformed using a variance stabilizing transformation;
assigning a hydration level to the subject based on the adjusted indicator concentration value, wherein the assigned hydration level is selected from a plurality of levels determined from a statistically treated set of indicator concentration values for a population of human subjects.

2. The method of claim 1, wherein the steps of transforming and calculating comprise using a variance stabilizing transformation selected from the group consisting of a square root function, a logarithmic transformation, and an arc-sin function to provide, respectively, the transformed indicator concentration value and the previously determined indicator concentration value.

3. The method of claim 1, wherein calculating comprises adjusting the transformed indicator concentration value by a previously determined indicator concentration value for the subject that has been logarithmically transformed.

4. The method of claim 3, wherein the logarithmically transformed indicator concentration value is a logarithmically transformed indicator concentration median value, a logarithmically transformed indicator concentration mean value or a logarithmically transformed indicator concentration single value.

5. The method of claim 4, wherein when the logarithmically transformed indicator concentration value is a median value or a mean value, it is based on a plurality of indicator concentration values from a single saliva sample from the subject or it is based on an indicator concentration value from a plurality of saliva samples from the subject.

6. The method of claim 1, wherein transforming comprises transforming the indicator concentration value with a logarithmic transformation selected from a log₂, log₁₀, or ln transform.

7. The method of claim 6, wherein transforming comprises calculating an adjusted indicator concentration value by subtracting from the adjusted indicator concentration value a log₂, log₁₀, or ln transformed baseline indicator concentration value for the subject.

8. The method of claim 7, wherein the logarithmically transformed baseline indicator concentration value is a logarithmically transformed median baseline indicator concentration value based on three or more saliva indicator concentration values from the subject.

9. The method of claim 8, wherein the logarithmically transformed median baseline indicator concentration value is a log₂ transformed median baseline value.

10. The method of claim 9, wherein the standard deviation of the three or more log₂ transformed median baseline indicator concentration values is less than 0.40.

11. The method of claim 1, further comprising adjusting the adjusted saliva indicator concentration value by an exercise adjustment factor if the saliva indicator concentration value is determined from a sample provided while the subject is exercising.

12. The method of claim 1, further comprising adjusting the adjusted saliva indicator concentration value by an elderly-person adjustment factor if the saliva indicator concentration value is determined from a sample provided by an elderly subject.

13. The method of claim 1, further comprising adjusting the adjusted saliva indicator concentration value by a medication adjustment factor if the saliva indicator concentration value is determined from a sample provided by a subject on a medication that affects saliva indicator concentration.

14. The method of claim 1, further comprising adjusting the adjusted saliva indicator concentration value by a disease-condition adjustment factor if the saliva indicator concentration value is determined from a sample provided by a person having a disease or condition that affects saliva indicator concentration.

15. The method of claim 11, wherein the adjusting comprises subtracting from the adjusted saliva indicator concentration value a standard deviation SD of the statistically treated set of indicator concentration values for the population of human subjects, to provide an exercise adjusted saliva indicator concentration value, and assigning a hydration level based on the exercise adjusted saliva indicator concentration value.

16. The method of claim 1, wherein the assigning comprises assigning a hydration level selected from a plurality of levels determined from a set of indicator concentration values for a population of human subjects, the set of indicator concentration values statistically treated using a statistical tool selected from the group consisting of linear regression analysis, analysis of variance, discriminant analysis and robust regression, and the hydration levels are taken as a standard deviation SD of the statistically treated set of indicator concentration values.

17. The method of claim 1, wherein the indicator concentration value is an osmolality value.

18. A method to determine a hydration level in a human subject, comprising:
determining a saliva osmolality value from a saliva sample from a human subject;
transforming the saliva osmolality value to determine a transformed saliva osmolality value;
calculating an adjusted saliva osmolality value by adjusting the transformed saliva osmolality value by a previously determined, transformed median baseline osmolality value for the subject; and
assigning a hydration level to the subject based on the adjusted saliva osmolality value, wherein the assigned hydration level is based on a standard deviation x of a statistically treated set of osmolality values for the subject, wherein an adjusted saliva osmolality value less than x defines a first hydration level, an adjusted saliva osmolality value equal to or greater than x and less than b·x defines a second hydration level, and an adjusted saliva osmolality value greater than or equal to b·x defines a third hydration level.

19. A method to evaluate hydration level in a subject, comprising:
determining a saliva osmolality value from a saliva sample from a human subject;
transforming the saliva osmolality value to a logarithmic value to determine a transformed saliva osmolality value;
assigning a hydration level to the subject based on the transformed saliva osmolality value, wherein the hydration level is selected from a plurality of hydration levels determined from a calibration standard curve of logarithmically transformed saliva osmolality values as a function of percent body weight loss.

20. The method of claim 19, wherein assigning comprises assigning a hydration level selected from a plurality of hydration levels determined from a calibration standard curve of saliva osmolality values logarithmically transformed log₂, log₁₀, or ln.
